(19) United States
(12) Patent Application Publication
(10) Pub. No.: US 2023/0113890 A1
(43) Pub. Date: Apr. 13, 2023

(54) VEGETATION OBSERVATION DEVICE, VEGETATION OBSERVATION SYSTEM, AND VEGETATION OBSERVATION METHOD

(71) Applicant: NEC Corporation, Minato-ku, Tokyo (JP)

(72) Inventor: Katsuhiro YUTANI, Tokyo (JP)

(73) Assignee: NEC Corporation, Minato-ku, Tokyo (JP)

(21) Appl. No.: 17/909,534

(22) PCT Filed: Mar. 19, 2021
(86) PCT No.: PCT/JP2021/011422

§ 371 Date: Sep. 6, 2022

(30) Foreign Application Priority Data
Mar. 27, 2020 (JP) 2020-058404

(51) Int. Cl. G01S 17/89; G01S 17/06; G01S 7/486; G01N 33/00

(52) U.S. Cl. CPC G01S 17/89; G01S 17/06; G01S 7/4868; G01N 33/0098

(57) ABSTRACT

In order to provide a vegetation observation device capable of stereographically observing the state of vegetation, this vegetation observation device is provided with: a vegetation model generation means for generating a vegetation model which is a three-dimensional model of a vegetation region, on the basis of laser reflected light generated when a laser beam to be applied to a light irradiation region including the vegetation region is reflected by vegetation in the vegetation region; and a vegetation observation means for observing vegetation in the vegetation region, on the basis of the vegetation model generated by the vegetation model generation means.

### TECHNICAL FIELD

The present invention relates to, for example, a vegetation observation device, a vegetation observation system, a vegetation observation method, and a storage medium storing therein a vegetation observation program that observe vegetation of an agricultural crop such as rice and a fruit tree.

### BACKGROUND ART

In recent years, a light detection and ranging (LiDAR) technique of emitting laser light to a target, analyzing reflected light from the target, and thereby detecting a property of the target or a distance to the target has been known.

For example, PTL 1 discloses a technique of recognizing growth status of an agricultural crop by using the LiDAR technique. PTL 2 discloses a technique relating to an inspection device that inspects vegetation.

### CITATION LIST

### Patent Literature

[PTL 1] International Publication No. WO2016/208415

[PTL 2] International Publication No. WO2016/009688

### SUMMARY OF INVENTION

### Technical Problem

However, in the invention described in PTL 1, growth status of an agricultural crop is recognized by use of a two-dimensional image capturing reflected light of laser light emitted by a laser radar device. Thus, there has been a problem that a state of vegetation (e.g., growth status or the like of an agricultural crop planted within a farm) is not able to be stereographically observed.

An object of the present invention is to provide a vegetation observation device and the like that are able to stereographically observe a state of vegetation.

### Solution to Problem

According to the present invention, a vegetation observation device includes:

a vegetation model generation means for generating a vegetation model being a three-dimensional model of a vegetation region, based on laser reflected light being light generated when laser light to be emitted to a light irradiation region including the vegetation region is reflected by vegetation within the vegetation region; and

a vegetation observation means for observing vegetation within the vegetation region, based on the vegetation model generated by the vegetation model generation means.

According to the present invention, a vegetation observation system includes:

a vegetation model generation means for generating a vegetation model being a three-dimensional model of a vegetation region, based on laser reflected light being light generated when laser light to be emitted to a light irradiation region including the vegetation region is reflected by vegetation within the vegetation region; and

a vegetation observation means for observing vegetation within the vegetation region, based on the vegetation model generated by the vegetation model generation means.

According to the present invention, a vegetation observation method includes:

generating a vegetation model being a three-dimensional model of a vegetation region, based on laser reflected light being light generated when laser light to be emitted to a light irradiation region including the vegetation region is reflected by vegetation within the vegetation region; and

observing vegetation within the vegetation region, based on the generated vegetation model.

According to the present invention, a storage medium stores therein a vegetation observation program that causes an information processing device to execute:

a step of generating a vegetation model being a three-dimensional model of a vegetation region, based on laser reflected light being light generated when laser light to be emitted to a light irradiation region including the vegetation region is reflected by vegetation within the vegetation region; and

a step of observing vegetation within the vegetation region, based on the generated vegetation model.

### Advantageous Effects of Invention

According to the present invention, it is possible to provide a vegetation observation device, a vegetation observation system, a vegetation observation method, and a storage medium storing therein a vegetation observation program that are able to stereographically observe a state of vegetation.

### BRIEF DESCRIPTION OF DRAWINGS

FIG. 1 is a block diagram illustrating a configuration example of a vegetation observation device according to a first example embodiment of the present invention.

FIG. 2 is a diagram for describing details of the vegetation observation device according to the first example embodiment of the present invention.

FIG. 3 is a diagram for describing details of the vegetation observation device according to the first example embodiment of the present invention.

FIG. 4 is a diagram for describing details of the vegetation observation device according to the first example embodiment of the present invention.

FIG. 5 is a flowchart illustrating an operation example of the vegetation observation device according to the first example embodiment of the present invention.

FIG. 6 is a block diagram illustrating a configuration of a modified example of the vegetation observation device according to the first example embodiment of the present invention.

FIG. 7 is a flowchart illustrating an operation of the modified example of the vegetation observation device according to the first example embodiment of the present invention.

FIG. 8 is a block diagram illustrating a configuration example of a vegetation observation device according to a second example embodiment of the present invention.

FIG. 9 is a flowchart illustrating an operation example of the vegetation observation device according to the second example embodiment of the present invention.

### EXAMPLE EMBODIMENT

### First Example Embodiment

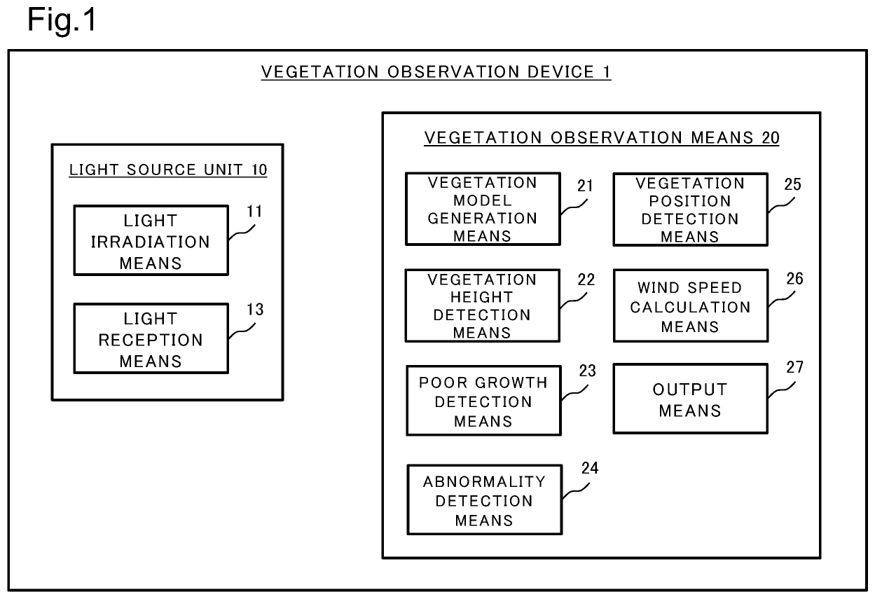

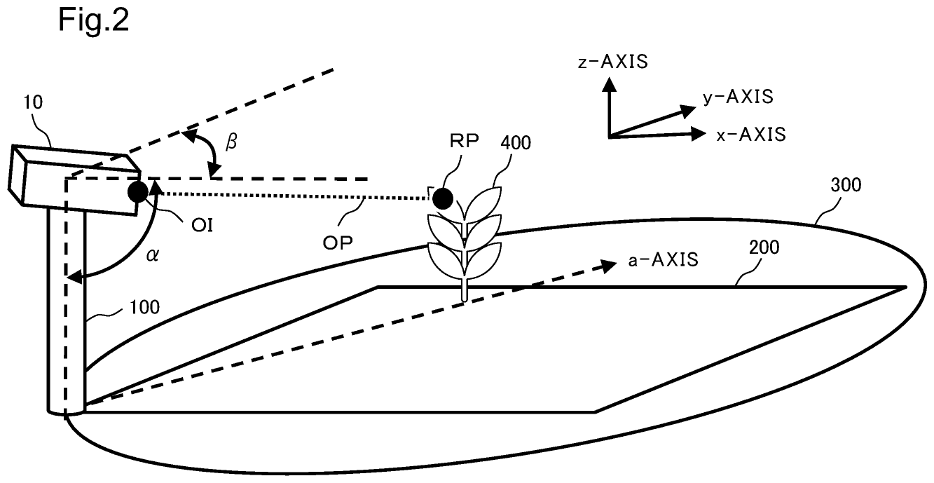

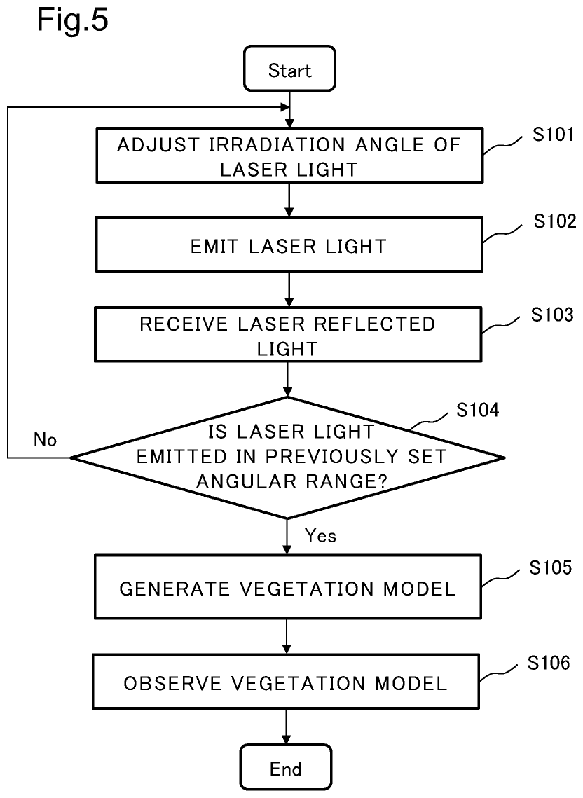

A vegetation observation device 1 according to a first example embodiment is described based on FIGS. 1, 2, 3, 4, and 5. FIG. 1 is a block diagram illustrating a configuration example of the vegetation observation device 1. FIGS. 2, 3, and 4 are diagrams for describing details of the vegetation observation device 1. FIG. 5 is a flowchart diagram for describing an operation example of the vegetation observation device 1.

A configuration of the vegetation observation device 1 is described. The vegetation observation device 1 includes a light source unit 10 and a vegetation observation means 20. Note that, the light source unit 10 and the vegetation observation means 20 are integrally provided in FIG. 1, but may be separate. The light source unit 10 and the vegetation observation means 20 are communicable with each other by a non-illustrated communication means.

The light source unit 10 includes a light irradiation means 11 and a light reception means 13.

The light irradiation means 11 emits laser light to a light irradiation region 300 including a vegetation region 200. Specifically, laser light is pulsed laser light. For example, as illustrated in FIGS. 2, 3, and 4, the light irradiation means 11 emits laser light from a light input/output terminal OI provided in the light source unit 10. Thereby, the emitted laser light propagates along an optical path OP, and enters a reflection point RP of vegetation 400 existing within the vegetation region 200. The optical path OP is a segment connecting the light input/output terminal OI and the reflection point RP. Herein, vegetation refers to a group of plants, for example, rice, a fruit tree or a tree. The vegetation region 200 refers to a region where a plant grows.

The light reception means 13 receives laser light (hereinafter, referred to as “laser reflected light”.) reflected by the vegetation 400 within the vegetation region 200. For example, in the example of FIGS. 2, 3, and 4, the light reception means 13 receives laser reflected light from the reflection point RP of the vegetation 400 via the optical path OP and the light input/output terminal OI. The light reception means 13 is able to receive pieces of laser reflected light from the different reflection points RP, by changing a direction in which the light source unit 10 emits laser light as described later.

Next, the vegetation observation means 20 is described. The vegetation observation means 20 includes a vegetation model generation means 21, a vegetation height detection means 22, a poor growth detection means 23, an abnormality detection means 24, a vegetation position detection means 25, a wind speed calculation means 26, and an output means 27. The vegetation observation means 20 observes vegetation within the vegetation region 200, based on a vegetation model.

The vegetation model generation means 21 is described. The vegetation model generation means 21 generates a vegetation model being a three-dimensional model of the vegetation region 200, based on laser reflected light received by the light reception means 13. The three-dimensional model is an aggregate of points positions of which are uniquely determined by a coordinate of an x-axis, a coordinate of a y-axis, and a coordinate of a z-axis. The three-dimensional model is, for example, a three-dimensional point group model.

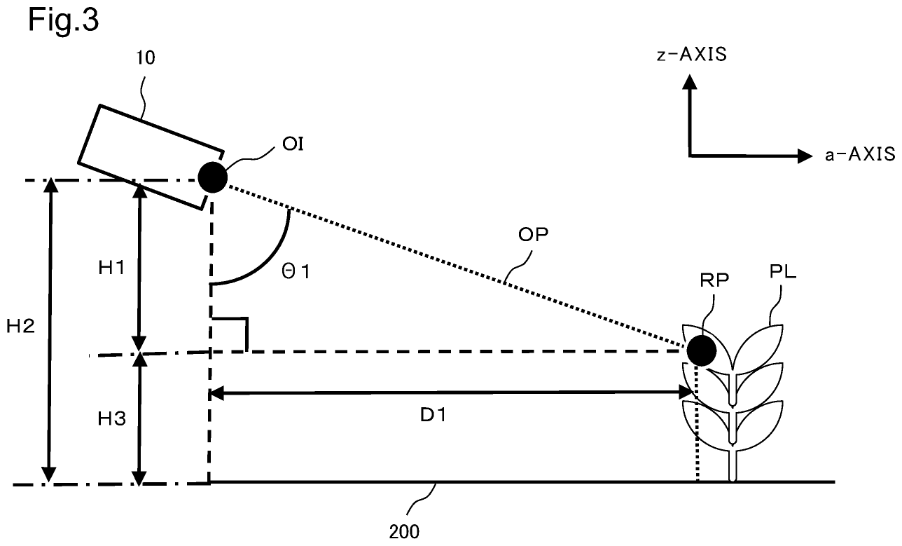

Herein, details of a generation method of a vegetation model are described by use of FIGS. 2, 3, and 4. FIG. 2 illustrates a position relation between the light source unit 10 and the vegetation region 200 by the x-axis, the y-axis, and the z-axis. FIG. 3 illustrates a position relation between the light source unit 10 and the vegetation region 200 by the z-axis and an a-axis. The a-axis is able to be acquired by performing orthogonal projection of the optical path OP on an xy plane.

The light source unit 10 inclines along an a-direction (an up-down direction relative to the xy plane) illustrated in FIG. 2, and thereby, the light irradiation means 11 is able to emit laser light at any angle θ1 as illustrated in FIG. 3. For example, as illustrated in FIG. 3, the angle θ1 is an angle of a corner formed by a straight line extending vertically downwards from the light input/output terminal OI of laser light and the optical path OP. The vegetation model generation means 21 is able to detect the angle θ1 by a non-illustrated gyro sensor or the like.

When determining a z-coordinate of the reflection point RP included in a three-dimensional model, the vegetation model generation means 21 derives a length of the optical path OP from a time of irradiation of laser light by the light irradiation means 11 to reception of laser reflected light by the light reception means 13 (hereinafter, referred to as a time t). Specifically, a length of the optical path OP is derived by dividing, by 2, a value resulting from multiplication of the time t by light speed. The vegetation model generation means 21 is able to calculate a difference (H1 in FIG. 3) between the z-coordinate of the light input/output terminal OI of laser light and the z-coordinate of the reflection point RP of the laser light by multiplying the length of the optical path OP by cos θ1. Thereby, the vegetation model generation means 21 acquires a relative position on the z-axis of the reflection point RP relative to the light input/output terminal OI.

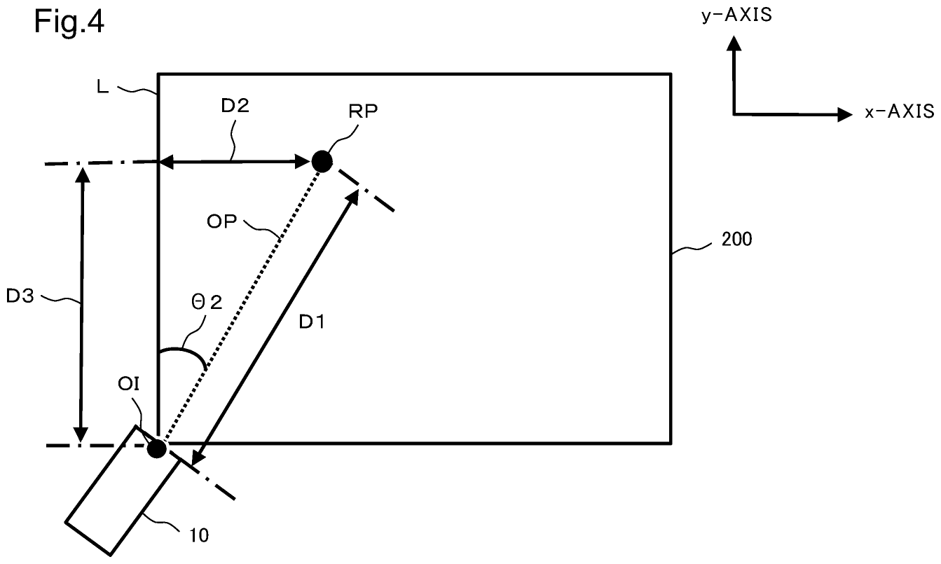

Furthermore, the vegetation model generation means 21 calculates a length of a segment D1 of the optical path OP projected on the xy plane, by multiplying a length of the optical path OP by sin θ1. The segment D1 is a segment connecting from the light input/output terminal OI of the laser light to the reflection point RP on the xy plane, as illustrated in FIG. 4.

The light source unit 10 inclines along a β-direction (a direction parallel to the xy plane) illustrated in FIG. 2, and thereby, the light irradiation means 11 is able to emit laser light at any angle θ2. For example, as illustrated in FIG. 4, the angle θ2 is an angle of a corner formed by a criterion line L set on the xy plane and the optical path OP. In the example illustrated in FIG. 4, the criterion line L is one side among sides constituting an outer periphery of the vegetation region 200. The vegetation model generation means 21 is able to detect the angle θ2 by a non-illustrated gyro sensor or the like.

The vegetation model generation means 21 multiplies the length of the segment D1 by sin θ2, and thereby derives a difference (D2 in FIG. 4) between an x-coordinate of the light input/output terminal OI and an x-coordinate of the reflection point RP. The vegetation model generation means 21 multiplies the length of the segment D1 by sin θ2, and thereby derives a difference (D3 in FIG. 4) between a y-coordinate of the light input/output terminal OI and a y-coordinate of the reflection point RP. Thereby, the vegetation model generation means 21 acquires a relative position on the x-axis and a relative position on the y-axis of the reflection point RP relative to the light input/output terminal OI. The vegetation model generation means 21 stores the acquired relative position on each of the axes in association with the angle θ1 and the angle θ2.

The light source unit 10 changes at least one of the angles θ1 and θ2, and thereby, laser light enters the reflection point RP at a different position. The light source unit 10 emits laser light according to a plurality of the predetermined angles θ1 and a plurality of the predetermined angle θ2, and thereby receives reflected laser light from a plurality of the reflection points RP within the light irradiation region 300. Thereby, the vegetation model generation means 21 is able to acquire a relative position on each axis, for each of a plurality of the reflection points RP within the vegetation region 200. The vegetation model generation means 21 generates a vegetation model by plotting a plurality of the reflection points RP on a three-dimensional model, based on a relative position of the reflection points RP to the light input/output terminal θ1.

The vegetation height detection means 22 detects a height of the vegetation 400 within the vegetation region 200. Specifically, the vegetation height detection means 22 detects a height of the vegetation 400, based on a vegetation height criterion point placed within the light irradiation region 300 and the generated vegetation model. The vegetation height criterion point is a criterion point to be a criterion of a height of the vegetation 400.

The vegetation height detection means 22 stores a criterion of a height (z-coordinate) for each coordinate on the xy plane of the light irradiation region 300. Herein, the vegetation height criterion point is placed within the light irradiation region 300. Then, the vegetation height detection means 22 previously stores, as a criterion coordinate, the z-coordinate (e.g., altitude (orthometric height) at the vegetation height criterion point. Note that, the vegetation height criterion point is a stone marker such as a triangulation point.

Herein, it is assumed that the z-coordinate of the vegetation height criterion point is 0 (zero). In this instance, a z-coordinate of the light input/output terminal OI is indicated by a distance (H2 in FIG. 3) from the vegetation height criterion point to the light input/output terminal OI. The vegetation height detection means 22 subtracts, from the z-coordinate (H2 in FIG. 3) of the light input/output terminal OI, a difference (H1 in FIG. 3) between the z-coordinate of the light input/output terminal OI of laser light and the z-coordinate of the reflection point RP of the laser light, and thereby acquires a height (H3 in FIG. 3) of the reflection point RP from the vegetation height criterion point. In this instance, the vegetation height detection means 22 acquires a height of the reflection point RP from the vegetation height criterion point as a height of the vegetation 400 at the reflection point RP. Note that, the vegetation height detection means 22 is described above as previously storing, as a criterion coordinate, the z-coordinate (e.g., altitude (orthometric height) at the vegetation height criterion point. However, the vegetation height detection means 22 is able to derive a distance between the vegetation height criterion point and the light input/output terminal OI, and set the distance as a z-coordinate of the light input/output terminal OI. At this moment, the light irradiation means 11 emits laser light to the vegetation height criterion point, and the light reception means 13 receives reflected light from the vegetation height criterion point. Then, the vegetation height detection means 22 derives, based on a time from the irradiation of the laser light to the reception of the laser reflected light and light speed, a linear distance between the vegetation height criterion point and the light input/output terminal OI. Further, the vegetation height detection means 22 multiplies, by a calculated value of the linear distance, a cosine of an angle of the light source unit 10 when the light source unit 10 is directed to the vegetation height criterion point, and derives a distance (H2 in FIG. 3) from the vegetation height criterion point to the light input/output terminal OI.

Note that, the vegetation height detection means 22 may add a parameter indicating a height of vegetation to a vegetation model. Specifically, the vegetation height detection means 22 may add, in a vegetation model in which a plurality of the reflection points RP are plotted on a three-dimensional space, a parameter indicating a height of the vegetation 400 at the reflection point RP for each of the reflection points RP.

The poor growth detection means 23 detects poor growth of the vegetation 400, based on a height of the vegetation 400. Specifically, when a height of the vegetation 400 at the reflection point RP is equal to or less than a previously set threshold value, the poor growth detection means 23 detects poor growth of the vegetation 400 at the reflection point RP. A threshold value may be previously determined based on a growth curve of the vegetation 400.

The poor growth detection means 23 detects poor growth of the vegetation 400, based on heights of a plurality of pieces of the vegetation 400. Specifically, the vegetation height detection means 22 detects heights of a plurality of pieces of the vegetation 400, from a plurality of the reflection points RP located within any region of the vegetation region 200. When, among a plurality of pieces of the acquired vegetation 400 (reflection points RP), pieces of vegetation heights of which are not more than a threshold value are more than a criterion, the poor growth detection means 23 detects poor growth (e.g., a low height of the vegetation 400 or a few leaves or fruits of the vegetation 400) in the region. In other words, the poor growth detection means 23 detects poor growth according to scarcity/density of pieces of the vegetation 400 heights of which are more than a threshold value. Note that, any region is uniquely determined by, for example, a predetermined range of an x-coordinate and a predetermined range of a y-coordinate on the xy plane of the light irradiation region 300.

The poor growth detection means 23 may detect poor growth of the vegetation 400, based on intensity of laser reflected light and a height of the vegetation 400. Laser light entering the reflection point RP is scattered according to a surface state of the reflection point RP. For example, as a surface state of the reflection point RP is smoother, an angle of scattering is narrower, and therefore, intensity of laser reflected light becomes strong. On the other hand, as a surface state of the reflection point RP is rougher, scattering is at a larger angle, and therefore, intensity of laser reflected light becomes weak. The poor growth detection means 23 utilizes this, and detects that the vegetation 400 is dead (poor growth) at the reflection point RP where intensity of laser reflected light is equal to or less than a threshold value, among the reflection points RP having heights being equal to or more than a predetermined value.

The poor growth detection means 23 may report, to outside of the vegetation observation device 1, poor growth of the vegetation 400 according to detection of the poor growth of the vegetation 400. Note that, not the poor growth detection means 23 but the output means 27 described later may report poor growth of the vegetation 400 to outside of the vegetation observation device 1 according to detection of the poor growth of the vegetation 400.

The abnormality detection means 24 detects occurrence of an abnormality in the vegetation 400, based on a temporal change of a height of the vegetation 400. Specifically, the abnormality detection means 24 compares a first vegetation model generated from reflected laser light received in a first period with a second vegetation model generated from reflected laser light received in a second period being later than the first period. The abnormality detection means 24 compares heights of pieces of the vegetation 400 at the reflection points RP x-coordinates and y-coordinates of which are correspondent or proximate to each other, among the reflection points RP included in the first vegetation model and the second vegetation model. When a height of the vegetation 400 at the reflection point RP included in the second vegetation model is lower, the abnormality detection means 24 detects an abnormality (e.g., collapse of the vegetation 400 due to intrusion of an animal) of the vegetation 400.

When an abnormality of the vegetation 400 is detected in an outer peripheral part of the vegetation region 200, the abnormality detection means 24 detects intrusion of an animal into the vegetation region 200. The abnormality detection means 24 previously stores a relative position, to the light input/output terminal OI, of the reflection point RP located in the outer peripheral part of the vegetation region 200. When an abnormality of the vegetation 400 is detected at the reflection point RP located in the outer peripheral part, the abnormality detection means 24 detects intrusion of an animal into the vegetation region 200.

The vegetation position detection means 25 detects a position of the vegetation 400 on a surface being perpendicular to a vertical direction. Specifically, the vegetation position detection means 25 detects a position of the reflection point RP on the xy plane as illustrated in FIGS. 2 and 4. For example, the vegetation position detection means 25 detects, as a position of the vegetation 400, a position of the reflection point RP on the xy plane, based on relative positions of the reflection point RP to the light input/output terminal OI on an x-axis and a y-axis.

The vegetation position detection means 25 detects, as a position of the vegetation 400, an absolute position of the vegetation 400 derived based on a position of a vegetation position criterion point placed within the light irradiation region 300 and a relative position of the vegetation 400 to an arrangement position of the light source unit 10. The vegetation position criterion point is a criterion point to be a criterion of a position within the vegetation region 200. Note that, the vegetation position criterion point may be the same as the above-described vegetation height criterion point.

Specifically, the vegetation position detection means 25 derives a relative position of the vegetation position criterion point to the light input/output terminal OI, as in the above-described method that derives a relative position of the reflection points RP to the light input/output terminal OI. For example, it is assumed that the vegetation position detection means 25 previously stores an absolute position of the vegetation position criterion point. The vegetation position detection means 25 derives an absolute position of the light input/output terminal OI, from the absolute position of the vegetation position criterion point and a relative position of the vegetation position criterion point to the light input/output terminal OI. Furthermore, the vegetation position detection means 25 derives an absolute position of the reflection point RP from the absolute position of the light input/output terminal OI and a relative position of the reflection points RP to the light input/output terminal OI. Then, the vegetation position detection means 25 detects the derived absolute position as a position of the vegetation 400. Note that, a detection method of an absolute position of the vegetation 400 is not limited to the method described above, and may be another method using an absolute position of the vegetation position criterion point.

Note that, an absolute position is indicated by, for example, latitude and longitude. The vegetation position criterion point may be used in common with the vegetation height criterion point described above. That is to say, a z-coordinate of the vegetation position criterion point may be a z-coordinate indicating an aboveground height of the light irradiation region 300, similarly to the vegetation height criterion point described above.

The wind speed calculation means 26 calculates a tremble speed of the vegetation 400, based on a difference of frequency between laser light and laser reflected light, and derives, based on the tremble speed, a wind speed of a wind applied to the vegetation 400. The wind speed calculation means 26 previously stores a frequency of laser light emitted by the light irradiation means 11. The light reception means 13 performs coherent detection of laser reflected light by use of local light emission of the same frequency as the laser light, and thereby detects a frequency of the laser reflected light. The wind speed calculation means 26 calculates, as a frequency shift amount by Doppler effect, a difference between a frequency of laser light and a frequency of laser reflected light. Furthermore, the wind speed calculation means 26 derives a movement speed of the reflection point RP from the frequency shift amount, and determines the movement speed as a tremble speed of the vegetation 400 of the reflection point RP.

Further, the wind speed calculation means 26 derives a wind speed from the derived tremble speed. For example, the wind speed calculation means 26 previously stores, in a non-illustrated memory, a table associating a tremble speed and a wind speed with each other. In this instance, the wind speed calculation means 26 detects, as a wind speed at the reflection point RP, a wind speed associated with the derived tremble speed. Note that, it is assumed that the table described above is previously produced by measuring an actual measurement value of a tremble speed and an actual measurement value of a wind speed. Generally, since there is a tendency that vegetation at a position where a wind speed is high does not grow easily, the vegetation observation device 1 is able to derive a wind speed of a wind applied to the vegetation 400, and thereby specify the vegetation 400 that easily results in poor growth.

The output means 27 outputs information relating to the vegetation 400 in the vegetation region 200 observed by the vegetation observation means 20. Specifically, the output means 27 outputs, to outside of the vegetation observation device 1, at least one of a vegetation model generated by the vegetation model generation means 21, a height of the vegetation 400 detected by the vegetation height detection means 22, poor growth of the vegetation 400 detected by the poor growth detection means 23, an abnormality of the vegetation 400 detected by the abnormality detection means 24, a position of the vegetation 400 detected by the vegetation position detection means 25, and a wind speed of a wind applied to the vegetation 400 calculated by the wind speed calculation means 26. Specifically, the output means 27 outputs information relating to the vegetation 400 via a non-illustrated display, speaker, or the like. Note that, the output means 27 may superimpose and display various kinds of information over a vegetation model. For example, the output means 27 may superimpose and display, over a vegetation model, a height of the vegetation 400 detected by the vegetation height detection means 22.

Next, an operation example of the vegetation observation device 1 is described by use of FIG. 5.

The light source unit 10 adjusts an irradiation angle of laser light (S101). For example, the light source unit 10 adjusts, to a predetermined angle, an angle θ1 illustrated in FIG. 3 and an angle θ2 illustrated in FIG. 4.

The light irradiation means 11 of the light source unit 10 emits laser light (S102). Thereby, the laser light is reflected at the reflection point RP of the vegetation 400.

The light reception means 13 of the light source unit 10 receives laser reflected light (S103). In this instance, a time t from the irradiation of the laser light to the reception of the reflected laser light is stored in a non-illustrated memory included in the vegetation observation device 1, in association with an irradiation angle of the laser light. Note that, in this instance, the light source unit 10 may store, in addition to the time t, intensity of the reflected laser light, and a difference of frequency between the laser light and the reflected laser light.

The light source unit 10 determines whether the laser light is emitted in a predetermined angular range (S104).

When the laser light is not emitted in the predetermined angular range (No of S104), the light source unit 10 adjusts an irradiation angle of the laser light (S101). For example, the light source unit 10 changes at least one of the angle θ1 illustrated in FIG. 3 and the angle θ2 illustrated in FIG. 4.

When the laser light is emitted in the predetermined angular range (Yes of S104), the vegetation model generation means 21 of the vegetation observation means 20 generates a vegetation model (S105). Specifically, the vegetation model generation means 21 generates a vegetation model according to the details of the generation method of a vegetation model described above.

The vegetation observation means 20 observes the vegetation model (S106). Herein, an example of the observation in the vegetation observation means 20 is as follows. A first example is detecting the vegetation 400 by the vegetation height detection means 22. A second example is detecting poor growth of the vegetation 400 by the poor growth detection means 23. A third example is detecting an abnormality of the vegetation 400 by the abnormality detection means 24. A fourth example is detecting a position of the vegetation 400 by the vegetation position detection means 25. A fifth example is calculating, by the wind speed calculation means 26, a wind speed of a wind applied to the vegetation 400. A sixth example is outputting, to outside of the vegetation observation device 1 by the output means 27, a detection or calculation in the above-described first to fifth examples. The operation example of the vegetation observation device 1 has been described above.

Note that, in the above description, it is assumed that the vegetation observation means 20 of the vegetation observation device 1 includes all of the vegetation model generation means 21, the vegetation height detection means 22, the poor growth detection means 23, the abnormality detection means 24, the vegetation position detection means 25, the wind speed calculation means 26, and the output means 27. However, the vegetation observation means 20 does not need to include all of the elements. Specifically, the vegetation observation means 20 may include at least one of the vegetation height detection means 22, the poor growth detection means 23, the abnormality detection means 24, the vegetation position detection means 25, the wind speed calculation means 26 and the output means 27, and the vegetation model generation means 21.

As above, the vegetation observation device 1 includes the vegetation model generation means 21 for generating, based on laser reflected light, a vegetation model being a three-dimensional model of the vegetation region 200. Herein, the laser reflected light is light generated when laser light to be emitted to the light irradiation region 300 including the vegetation region 200 is reflected by the vegetation 400 within the vegetation region 200. The vegetation observation device 1 includes the vegetation observation means 20 for observing vegetation within the vegetation region 200, based on the vegetation model generated by the vegetation model generation means 21. The vegetation observation means 20 is, for example, the vegetation height detection means 22, the poor growth detection means 23, the abnormality detection means 24, the vegetation position detection means 25, the wind speed calculation means 26, or the output means 27.

As above, the vegetation model generation means 21 generates a vegetation model being a three-dimensional model of the vegetation region 200. Thereby, according to the vegetation observation device 1, a state of the vegetation 400 is able to be stereographically observed by use of a vegetation model being a three-dimensional model of the vegetation region 200.

The vegetation observation device 1 includes the vegetation height detection means 22 for detecting a height of the vegetation 400 within the vegetation region 200. Thereby, the vegetation observation device 1 is able to stereographic ally observe a height of the vegetation 400.

The vegetation observation device 1 includes the vegetation height detection means 22 for detecting a height of the vegetation 400 within the vegetation region 200, based on a vegetation height criterion point being a criterion point to be a criterion of a height of the vegetation 400, and being placed within the light irradiation region 300, and a vegetation model generated by the vegetation model generation means 21. As above, in the vegetation observation device 1, the vegetation model generation means 21 generates a vegetation model, and then, the vegetation height detection means 22 detects a height of the vegetation 400 within the vegetation region 200. Thereby, according to the vegetation observation device 1, a height of the vegetation 400 is able to be stereographically observed.

The vegetation observation device 1 includes the poor growth detection means 23 for detecting poor growth of the vegetation 400, based on a height of the vegetation 400. The poor growth detection means 23 detects poor growth of the vegetation 400, based on a previously set threshold value for a height of the vegetation 400, and a height of the vegetation 400. Thereby, the vegetation observation device 1 is able to detect, as the vegetation 400 being poor in growth, the vegetation 400 being low in height within the vegetation region 200.

The poor growth detection means 23 of the vegetation observation device 1 detects poor growth of the vegetation 400, based on intensity of laser reflected light and a height of the vegetation 400. Thereby, the vegetation 400 being rough in a surface state for such a reason as being dead is able to be detected as being poor in growth.

The vegetation observation device 1 includes the abnormality detection means 24 for detecting an abnormality of the vegetation 400, based on a temporal change of a height of the vegetation 400. Thereby, when a height of the vegetation 400 becomes low, the vegetation observation device 1 is able to detect, as an abnormality in the vegetation 400, collapse of the vegetation 400 due to, for example, intrusion of an animal.

The vegetation observation device 1 further includes the light source unit 10 including the light irradiation means 11 for emitting laser light to the vegetation region 200, and the light reception means 13 for receiving laser reflected light. The vegetation model generation means 21 generates a vegetation model, based on laser reflected light received by the light reception means 13.

The vegetation observation device 1 includes the vegetation position detection means 25 for detecting a position of the vegetation 400 on a surface being perpendicular to a vertical direction. For example, the vegetation position detection means 25 detects, as a position of the vegetation 400, a relative position of the vegetation 400 to an arrangement position of the light source unit 10. The vegetation position detection means 25 detects, as a position of the vegetation 400, an absolute position of the vegetation 400 derived based on a position of a vegetation position criterion point placed within the light irradiation region 300, and a relative position of the vegetation 400 to an arrangement position of the light source unit 10. Note that, the vegetation position criterion point is a criterion point to be a criterion of a position within the vegetation region 200. Thereby, the vegetation observation device 1 is able to determine a position of the vegetation 400 on the vegetation region 200.

The vegetation observation device 1 includes the wind speed calculation means 26 for calculating a tremble speed of the vegetation 400, based on a difference of frequency between laser light and laser reflected light, and deriving, based on the tremble speed, a wind speed of a wind applied to the vegetation 400. Thereby, the vegetation observation device 1 is able to detect a wind speed within the vegetation region 200.

The vegetation observation device 1 further includes the output means 27 for outputting information relating to the vegetation 400 within the vegetation region 200 observed by the vegetation observation means 20. Thereby, the vegetation observation device 1 is able to output information relating to the vegetation 400 to outside of the vegetation observation device 1.

The vegetation height detection means 22 superimposes a height of the vegetation 400 over a vegetation model generated by the vegetation model generation means 21. Thereby, the vegetation observation device 1 is able to display, as one three-dimensional model, a height of the vegetation 400 and a vegetation model.

When poor growth of the vegetation 400 is detected, the poor growth detection means 23 reports the poor growth of the vegetation 400. Thereby, the vegetation observation device 1 is able to immediately report occurrence of poor growth.

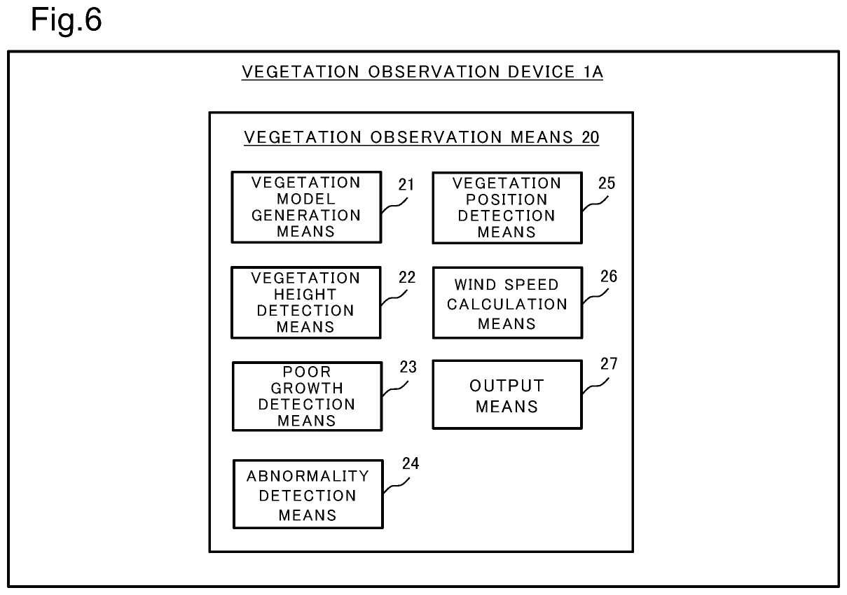

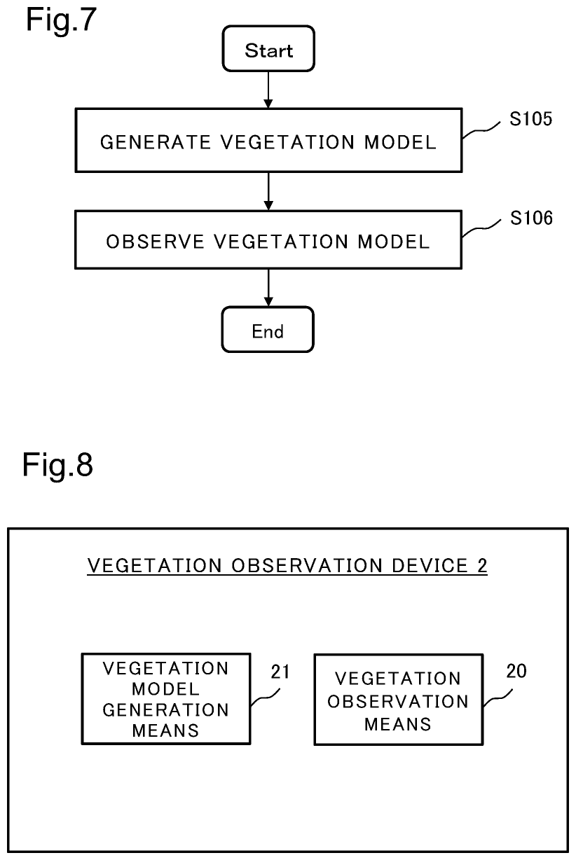

The vegetation observation device 1 has been described above. Next, a vegetation observation device 1A is described by use of FIGS. 6 and 7. FIG. 6 is a block diagram illustrating a configuration example of the vegetation observation device 1A. FIG. 7 is a flowchart illustrating an operation example of the vegetation observation device 1A.

The vegetation observation device 1A is a modified example of the vegetation observation device 1. Each component included in the vegetation observation device 1A includes a similar function and configuration to a component included in the vegetation observation device 1. On the other hand, the vegetation observation device 1A differs from the vegetation observation device 1 in not including the light source unit 10. The vegetation observation device 1A acquires, from an external device, information relating to laser reflected light being light generated when laser light to be emitted to the light irradiation region 300 including the vegetation region 200 is reflected by the vegetation 400 within the vegetation region 200.

An operation of the vegetation observation device 1A is described by use of FIG. 7. As illustrated in FIG. 7, the vegetation observation device 1A performs only S105 and S106 among the operations S101 to S106 of the vegetation observation device 1. The vegetation observation device 1A exerts an effect similar to an effect of the vegetation observation device 1.

### Second Example Embodiment

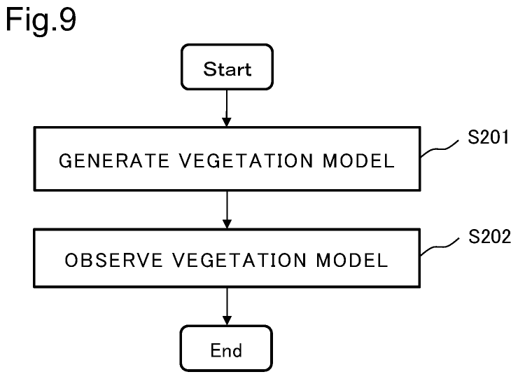

A vegetation observation device 2 according to a second example embodiment is described by use of FIGS. 8 and 9. FIG. 8 is a block diagram illustrating a configuration example of the vegetation observation device 2. FIG. 9 is a flowchart illustrating an operation example of the vegetation observation device 2.

As illustrated in FIG. 8, the vegetation observation device 2 includes a vegetation model generation means 21 and a vegetation observation means 20. For example, the vegetation observation device 2 acquires, by an external device, information relating to laser reflected light being light generated when laser light to be emitted to a light irradiation region including a vegetation region is reflected by vegetation within the vegetation region.

The vegetation model generation means 21 generates, based on laser reflected light, a vegetation model being a three-dimensional model of the vegetation region.

The vegetation observation means 20 observes vegetation within the vegetation region, based on the vegetation model generated by the vegetation model generation means 21. The vegetation observation means 20 is achieved by including, for example, a vegetation height detection means 22, a poor growth detection means 23, an abnormality detection means 24, a vegetation position detection means 25, a wind speed calculation means 26, or an output means 27 that constitutes the vegetation observation means 20 shown in the first example embodiment.

Next, an operation example of the vegetation observation device 2 is described by use of FIG. 9.

The vegetation model generation means 21 generates, based on laser reflected light, a vegetation model being a three-dimensional model of a vegetation region (S201).

The vegetation observation means 20 observes vegetation within the vegetation region, based on the vegetation model generated by the vegetation model generation means 21 (S202). Specifically, for example, the vegetation observation means 20 detects a height of vegetation included in the vegetation model, similarly to the vegetation height detection means 22 in the vegetation observation device 1 described in the first example embodiment. The vegetation observation device 2 has been described above.

As above, the vegetation model generation means 21 generates a vegetation model being a three-dimensional model of the vegetation region 200. Thereby, according to the vegetation observation device 1, a state of the vegetation 400 is able to be stereographically observed by use of a vegetation model being a three-dimensional model of the vegetation region 200.

Note that, the vegetation model generation means 21 and the vegetation observation means 20 may be provided in different devices, and then operate as one vegetation observation system.

Some or all of the above-described example embodiments can also be described as, but are not limited to, the following supplementary notes.

### (Supplementary Note 1)

A vegetation observation device including:

a vegetation model generation means for generating a vegetation model being a three-dimensional model of a vegetation region, based on laser reflected light being light generated when laser light to be emitted to a light irradiation region including the vegetation region is reflected by vegetation within the vegetation region; and

a vegetation observation means for observing vegetation within the vegetation region, based on the vegetation model generated by the vegetation model generation means.

### (Supplementary Note 2)

The vegetation observation device according to supplementary note 1, wherein the vegetation observation means includes a vegetation position detection means for detecting a position of the vegetation on a surface being perpendicular to a vertical direction.

### (Supplementary Note 3)

The vegetation observation device according to supplementary note 1 or 2, further including a light source unit including a light irradiation means for emitting the laser light to the vegetation region, and a light reception means for receiving the laser reflected light, wherein

the vegetation model generation means generates the vegetation model, based on the laser reflected light received by the light reception means.

### (Supplementary Note 4)

The vegetation observation device according to supplementary note 3, wherein the vegetation observation means includes a vegetation position detection means for detecting, as a position of the vegetation, a relative position of the vegetation to an arrangement position of the light source unit.

### (Supplementary Note 5)

The vegetation observation device according to supplementary note 4, wherein the vegetation position detection means detects, as a position of the vegetation, an absolute position of the vegetation being derived based on a position of a vegetation position criterion point being a criterion point to be a criterion of a position within the vegetation region and being placed within the light irradiation region, and a relative position of the vegetation to an arrangement position of the light source unit.

### (Supplementary Note 6)

The vegetation observation device according to any one of supplementary notes 1 to 5, wherein the vegetation observation means includes a vegetation height detection means for detecting a height of the vegetation within the vegetation region.

### (Supplementary Note 7)

The vegetation observation device according to supplementary note 6, wherein the vegetation height detection means adds information indicating a height of the vegetation being detected by the vegetation height detection means, to the vegetation model generated by the vegetation model generation means.

### (Supplementary Note 8)

The vegetation observation device according to supplementary note 6 or 7, wherein the vegetation height detection means detects a height of the vegetation within the vegetation region, based on a vegetation height criterion point being a criterion point to be a criterion of a height of the vegetation and being placed within the light irradiation region, and the vegetation model generated by the vegetation model generation means.

### (Supplementary Note 9)

The vegetation observation device according to any one of supplementary notes 6 to 8, wherein the vegetation observation means includes a poor growth detection means for detecting poor growth of the vegetation, based on a height of the vegetation.

### (Supplementary Note 10)

The vegetation observation device according to supplementary note 9, wherein the poor growth detection means detects poor growth of vegetation in the vegetation region, based on heights of a plurality of pieces of the vegetation.

### (Supplementary Note 11)

The vegetation observation device according to supplementary note 9 or 10, wherein the poor growth detection means detects poor growth of the vegetation, based on a previously set threshold value for a height of the vegetation and a height of the vegetation.

### (Supplementary Note 12)

The vegetation observation device according to any one of supplementary notes 9 to 11, wherein the poor growth detection means detects poor growth of the vegetation, based on intensity of the laser reflected light and a height of the vegetation.

### (Supplementary Note 13)

The vegetation observation device according to any one of supplementary notes 9 to 12, wherein, when poor growth of the vegetation is detected, the poor growth detection means reports the poor growth of the vegetation to outside.

### (Supplementary Note 14)

The vegetation observation device according to any one of supplementary notes 6 to 13, wherein the vegetation observation means includes an abnormality detection means for detecting an abnormality of the vegetation, based on a temporal change of a height of the vegetation.

### (Supplementary Note 15)

The vegetation observation device according to supplementary note 14, wherein, when an abnormality of the vegetation is detected in an outer peripheral part of the vegetation region, the abnormality detection means detects intrusion of an animal into the vegetation region.

### (Supplementary Note 16)

The vegetation observation device according to any one of supplementary notes 1 to 15, wherein the vegetation observation means includes a wind speed calculation means for calculating a tremble speed of the vegetation, based on a difference of frequency between the laser light and the laser reflected light, and deriving, based on the tremble speed, a wind speed of a wind applied to the vegetation.

### (Supplementary Note 17)

The vegetation observation device according to any one of supplementary notes 1 to 16, further including an output means for outputting information relating to vegetation within the vegetation region being observed by the vegetation observation means.

### (Supplementary Note 18)

A vegetation observation system including:

a vegetation model generation means for generating a vegetation model being a three-dimensional model of a vegetation region, based on laser reflected light being light generated when laser light to be emitted to a light irradiation region including the vegetation region is reflected by vegetation within the vegetation region; and

a vegetation observation means for observing vegetation within the vegetation region, based on the vegetation model generated by the vegetation model generation means.

### (Supplementary Note 19)

A vegetation observation method including:

generating a vegetation model being a three-dimensional model of a vegetation region, based on laser reflected light being light generated when laser light to be emitted to a light irradiation region including the vegetation region is reflected by vegetation within the vegetation region; and

observing vegetation within the vegetation region, based on the generated vegetation model.

### (Supplementary Note 20)

A storage medium storing therein a vegetation observation program that causes an information processing device to execute:

a step of generating a vegetation model being a three-dimensional model of a vegetation region, based on laser reflected light being light generated when laser light to be emitted to a light irradiation region including the vegetation region is reflected by vegetation within the vegetation region; and

a step of observing vegetation within the vegetation region, based on the generated vegetation model.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

This application is based upon and claims the benefit of priority from Japanese patent application No. 2020-058404, filed on Mar. 27, 2020, the disclosure of which is incorporated herein in its entirety by reference.

### REFERENCE SIGNS LIST

1, 1A, 2 Vegetation observation device
10 Light source unit
11 Light irradiation means
13 Light reception means
20 Vegetation observation means
21 Vegetation model generation means
22 Vegetation height detection means
23 Poor growth detection means
24 Detection means
25 Vegetation position detection means
26 Wind speed calculation means
27 Output means

What is claimed is:

1. A vegetation observation device comprising:
a vegetation model generator configured to generate a vegetation model being a three-dimensional model of a vegetation region, based on laser reflected light being light generated when laser light to be emitted to a light irradiation region including the vegetation region is reflected by vegetation within the vegetation region; and
a vegetation observer configured to observe vegetation within the vegetation region, based on the vegetation model generated by the vegetation model generator.

2. The vegetation observation device according to claim 1, wherein the vegetation observer includes a vegetation position detector configured to detect a position of the vegetation on a surface being perpendicular to a vertical direction.

3. The vegetation observation device according to claim 1, further comprising a light source including a light irradiator configured to emit the laser light to the vegetation region and light receiver configured to receive the laser reflected light, wherein
the vegetation model generator generates the vegetation model, based on the laser reflected light received by the light receiver.

4. The vegetation observation device according to claim 3, wherein the vegetation observer includes a vegetation position detector configured to detect, as a position of the vegetation, a relative position of the vegetation to an arrangement position of the light source.

5. The vegetation observation device according to claim 4, wherein the vegetation position detector detects, as a position of the vegetation, an absolute position of the vegetation being derived based on a position of a vegetation position criterion point being a criterion point to be a criterion of a position within the vegetation region and being placed within the light irradiation region, and a relative position of the vegetation to an arrangement position of the light source.

6. The vegetation observation device according to claim 1, wherein the vegetation observer includes a vegetation height detector configured to detect a height of the vegetation within the vegetation region.

7. The vegetation observation device according to claim 6, wherein the vegetation height detector adds information indicating a height of the vegetation being detected by the vegetation height detector, to the vegetation model generated by the vegetation model generator.

8. The vegetation observation device according to claim 6, wherein the vegetation height detector detects a height of the vegetation within the vegetation region, based on a vegetation height criterion point being a criterion point to be a criterion of a height of the vegetation and being placed within the light irradiation region, and the vegetation model generated by the vegetation model generator.

9. The vegetation observation device according to claim 6, wherein the vegetation observer includes a poor growth detector configured to detect poor growth of the vegetation, based on a height of the vegetation.

10. The vegetation observation device according to claim 9, wherein the poor growth detector detects poor growth of vegetation in the vegetation region, based on heights of a plurality of pieces of the vegetation.

11. The vegetation observation device according to claim 9, wherein the poor growth detector detects poor growth of the vegetation, based on a previously set threshold value for a height of the vegetation and a height of the vegetation.

12. The vegetation observation device according to claim 9, wherein the poor growth detector detects poor growth of the vegetation, based on intensity of the laser reflected light and a height of the vegetation.

13. The vegetation observation device according to claim 9, wherein, when poor growth of the vegetation is detected, the poor growth detector reports the poor growth of the vegetation to outside.

14. The vegetation observation device according to claim 6, wherein the vegetation observer includes an abnormality detector configured to detect an abnormality of the vegetation, based on a temporal change of a height of the vegetation.

15. The vegetation observation device according to claim 14, wherein, when the abnormality of the vegetation is detected in an outer peripheral part of the vegetation region, the abnormality detector detects intrusion of an animal into the vegetation region.

16. The vegetation observation device according to claim 1, wherein the vegetation observer includes a wind speed calculator configured to calculate a tremble speed of the vegetation, based on a difference of frequency between the laser light and the laser reflected light, and deriving, based on the tremble speed, a wind speed of a wind applied to the vegetation.

17. The vegetation observation device according to claim 1, further comprising output portion configured to output information relating to vegetation within the vegetation region being observed by the vegetation observer.

18. A vegetation observation system comprising:
a vegetation model generator configured to generate a vegetation model being a three-dimensional model of a vegetation region, based on laser reflected light being light generated when laser light to be emitted to a light irradiation region including the vegetation region is reflected by vegetation within the vegetation region; and
a vegetation observer configured to observe vegetation within the vegetation region, based on the vegetation model generated by the vegetation model generator.

19. A vegetation observation method comprising:
generating a vegetation model being a three-dimensional model of a vegetation region, based on laser reflected light being light generated when laser light to be emitted to a light irradiation region including the vegetation region is reflected by vegetation within the vegetation region; and
observing vegetation within the vegetation region, based on the generated vegetation model.

20. (canceled)
